(19) United States
(12) Patent Application Publication
(10) Pub. No.: US 2024/0398211 A1
(43) Pub. Date: Dec. 5, 2024

(54) AN ENDOSCOPE

(71) Applicant: AMBU A/S, Ballerup (DK)

(72) Inventors: Victor SILBERMANN, Them (DK); Alistair David MORTON, Kastrup (DK)

(73) Assignee: AMBU A/S, Ballerup (DK)

(21) Appl. No.: 18/696,051

(22) PCT Filed: Sep. 22, 2022
(86) PCT No.: PCT/EP2022/076418

§ 371 Date: Mar. 27, 2024

(30) Foreign Application Priority Data
Sep. 28, 2021 (DK) PA 2021 70470

(51) Int. Cl. A61B 1/00; A61B 1/05; A61B 90/00

(52) U.S. Cl. CPC A61B 1/00128; A61B 1/00018; A61B 1/00045; A61B 1/00066; A61B 1/00114; A61B 1/05; A61B 90/08; A61B 2090/0807

(57) ABSTRACT

An endoscope (1) including a handle (5) including a housing part and a insertion cord (6a) coupled to the handle (5) by means of a rotary coupling mechanism allowing rotation about a rotation axis. The coupling mechanism includes a bushing having a through-aperture defining an inner bushing wall surface adapted to receive a connection member and an outer bushing wall surface adapted to engage an inner handle surface in the handle. A connection member is received in the through-aperture and has a wall defining a through passage between a first end and a second end. The first end is attached to an insertion cord sub-assembly, and the second end is rotatably coupled to a working channel part within the handle (5).

### CROSS-REFERENCE TO RELATED APPLICATIONS

This application is a National Stage entry under 35 U.S.C. § 371 of International Application No. PCT/EP2022/076418, filed Sep. 22, 2022, which claims priority from and the benefit of Danish Patent Application No. PA 2021 70470, filed Sep. 28, 2021; the disclosures of said applications are incorporated by reference herein in their entirety.

### TECHNICAL FIELD

The present disclosure relates to an endoscope, in particular but not exclusively a single use insertion endoscope with a rotatable insertion cord. More specifically, the present disclosure relates to an endoscope comprising a proximal handle comprising a housing part and a distal insertion cord coupled to said proximal handle by means of a rotary coupling mechanism allowing rotation about a rotation axis.

### BACKGROUND

In general, an endoscope comprises an operating handle at the proximal end and an insertion cord extending from the handle towards the distal end. The handle is adapted to be held by an operator and inter alia comprises externally protruding operating members connected to internal control gear allowing the operator to control the movement of a controllable bending section at the distal end of the insertion cord, while advancing the distal end of the insertion cord to a desired location e.g. within a body cavity of a person. By means of an attached monitoring device, such as a monitor with a display screen, the location to which the distal end has been advanced may be inspected using the endoscope.

The controllable bending section is normally an articulated section at the distal tip of the insertion cord that can be controlled by the operator from the handle, allowing the operator to advance the distal tip of the endoscope to a desired location by means of a series of actions involving inter alia bending the bending section in a desired direction, advancing the insertion cord and turning the insertion cord by turning the handle which is rigidly connected thereto. Having negotiated a tortuous path of bends and turns to a location of interest, the operator may end up in an awkward or ergonomically unfavourable body position.

This drawback may be overcome if the insertion cord is not rigidly connected to the handle, but may be turned with respect thereto. This allows the operator to turn the handle into a more convenient and ergonomically favourable position once the insertion cord is in place. It will also allow for an additional degree of freedom because the operator may choose to turn only the insertion cord rather than both the handle and the insertion cord when navigating during insertion.

In this respect U.S. Pat. No. 9,307,891 discloses an endoscope with a handle and an insertion cord that may be turned between a number of fixed latching positions with respect to the handle. There are no means to prevent turning the insertion cord too much with respect to the handle, just as statement that it should not be turned more than 180° in order not to break the inner workings i.e. the cables etc. running from the handle into the insertion cord. JP2004-305413 also discloses an endoscope with a handle and an insertion cord that may be turned with respect to the handle. The turning angle is limited by a stop so as not to twist and damage the fibre bundle running from the handle into the insertion cord when turning the insertion cord with respect to the handle.

In recent years single use or disposable endoscopes that are used on one patient only before being thrown away have gained substantially in popularity as compared to traditional endoscopes that were reused after difficult and costly cleaning and sterilisation. Evidently, if an endoscope is single use it needs to be manufactured in a cost-efficient manner, meaning that the constituent parts should have low cost, and that the assembly thereof should be simple so as not incur unnecessary time consumption.

In this respect, the prior art couplings do not render themselves to cost-efficient assembly as it will be difficult to thread the flexible bundle of e.g. electrical supply wires and signals cables, pull wires, optical fibres, etc. forming part of a sub-assembly though these coupling mechanisms between the handle and the insertion cord.

Moreover, in use, the coupling should give the operator good tactile feedback. That is to say sufficient friction to avoid inadvertent turning of the insertion cord but not so much that it prevents smooth manipulation by the average operator.

Also, the cable bundle should be protected from damaging from e.g. twisting the insertion cord too much with respect to the handle.

### BRIEF DESCRIPTION OF THE DISCLOSURE

Based on this it is the object of providing an endoscope having an insertion tube that may be turned with respect to the handle, and at the same time overcomes the above drawbacks.

According to a first aspect of the disclosure, this object is achieved by an endoscope comprising a proximal handle comprising a housing part and a distal insertion cord coupled to said proximal handle by means of a rotary coupling mechanism allowing rotation about a rotation axis, said coupling mechanism comprising a bushing having a through aperture defining an inner bushing wall surface adapted to receive a connection member and an outer bushing wall surface adapted to engage an inner handle surface in said handle, a connection member received in said through aperture and having a wall defining a through passage between a first end and a second end, wherein said first end is attached to an insertion cord sub-assembly, and said second end is rotatably coupled to a working channel part within said handle, wherein a lateral exit opening is provided in said wall between said first end and said second end comprises, and wherein a slit is provided in said wall from said second end to said exit opening.

This strongly facilitates the assembly of the endoscope because the entire insertion cord may be made or prefabricated as a separate insertion cord subassembly with all cables and wires extending from the proximal end. When attaching the proximal end of the insertion cord subassembly to the connection member, the cables and wires need not be passed axially through any ports or apertures but may just be passed laterally through the slit and emerge from the lateral exit opening. In other words, the cables and wires need not be threaded with the entire length with which they extend from the proximal end of the insertion cord subassembly through an eyelet. Rather, only a short length of the wires and cables need to be passed laterally through the slit which is a far easier procedure.

According to a preferred embodiment according to the first aspect of the disclosure, the inner bushing wall surface comprises a rib protruding into said slit. Once the bushing has been passed over the insertion cord from the distal end this rib closes the slit in the connection member and keep the cables and wires centred between the distal end of the connection member and the lateral exit opening.

According to an embodiment according to the first aspect of the disclosure, the coupling mechanism further comprises an annular gripping member having an external circumferential gripping surface and an inner gripping member surface, where the inner gripping member surface comprises a first inner gripping member surface part in engagement with said outer bushing wall surface and a second inner gripping member surface part rotatably engaging the exterior of said housing part. This allows a firm grip on the rotary part without having to grip the bushing wall itself which would normally be made of a low friction material less suitable for gripping.

According to an embodiment according to the first aspect of the disclosure, the endoscope further comprises a locking clip securing said bushing against axial motion with respect to the housing part along said rotation axis.

According to an embodiment, the locking clip is made from the same material as the bushing, so as to keep friction low.

According to an embodiment according to the first aspect of the disclosure, the second inner gripping member surface part comprises at least one position reference member adapted to mutually engage a complementary position reference member on the exterior of said housing part. This allows for temporary rest in a neutral centre position and tactile feedback to the operator when moving from the neutral centre position.

According to a further preferred embodiment according to the first aspect of the disclosure, the complementary position reference member is provided on a spring member provided integrally with the housing part, said spring member extending in a circumferential direction at the distal end of the housing part.

### BRIEF DESCRIPTION OF FIGURES

The disclosure will now be made in greater details based on non-limiting exemplary embodiments and with reference to the drawings, on which:

FIG. 1 shows a system comprising an endoscope according to the disclosure and a display device to which the endoscope may be connected,

FIG. 2 shows an exploded view of the endoscope of the system in FIG. 1,

FIG. 2A shows an exploded view of the handle part of the endoscope of FIG. 2 in greater detail and together with a connection cable,

FIG. 2B shows an exploded view of the insertion tube part of the endoscope of FIG. 2 in greater detail,

FIG. 3 shows the main handle housing part of FIGS. 2 and 2A in even greater detail,

FIG. 4 shows the insertion tube part as an assembled insertion tube sub-assembly,

FIGS. 5 and 6 show orthogonal views of the insertion tube sub-assembly and parts of the rotary coupling mechanism,

FIG. 7 shows an isometric view of a connection member,

FIG. 8 shows the connection of a connection member to a working channel tool insertion part in partial cross-section, and

FIG. 9 shows a cross-section of the working channel tool insertion part taken along the line IX-IX in FIG. 8, FIG. 10 shows an isometric view from the distal end of a bushing

FIG. 11 is a longitudinal section in isometry from the opposite end of the bushing of FIG. 10,

FIG. 12 is a longitudinal section of the bushing taken along the line XII-XII in FIG. 11

FIG. 13 shows an isometric view form the distal end of a gripping member,

FIG. 14 shows an isometric view form the proximal end of the gripping member of FIG. 13,

FIGS. 15 and 16 show orthogonal cross-sectional views of the section of endoscope with the assembled rotary coupling mechanism,

FIG. 17 shows a tool used in the mounting of the working channel tube, and

FIG. 18 shows a detail of the coupling mechanism in cross section with the tool in place.

### DETAILED DESCRIPTION

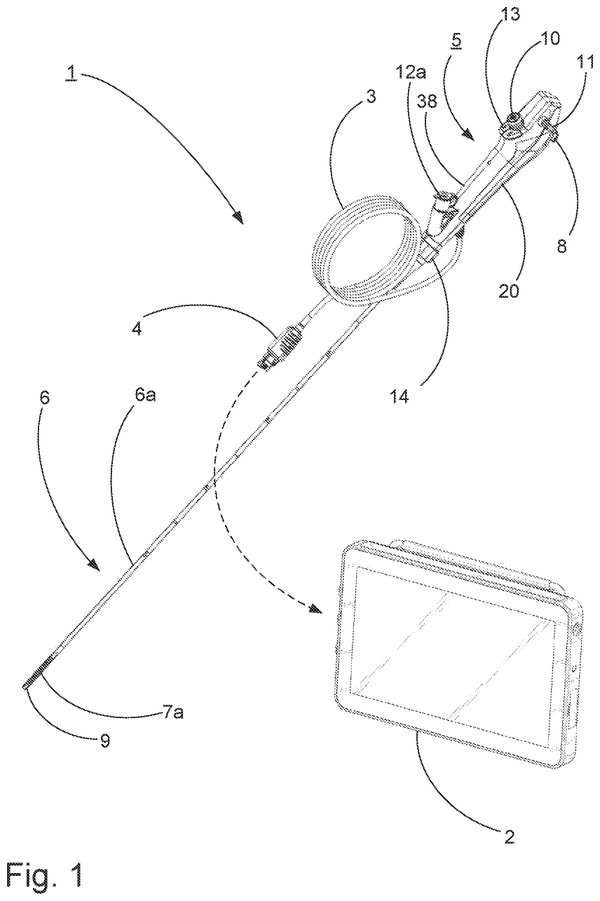

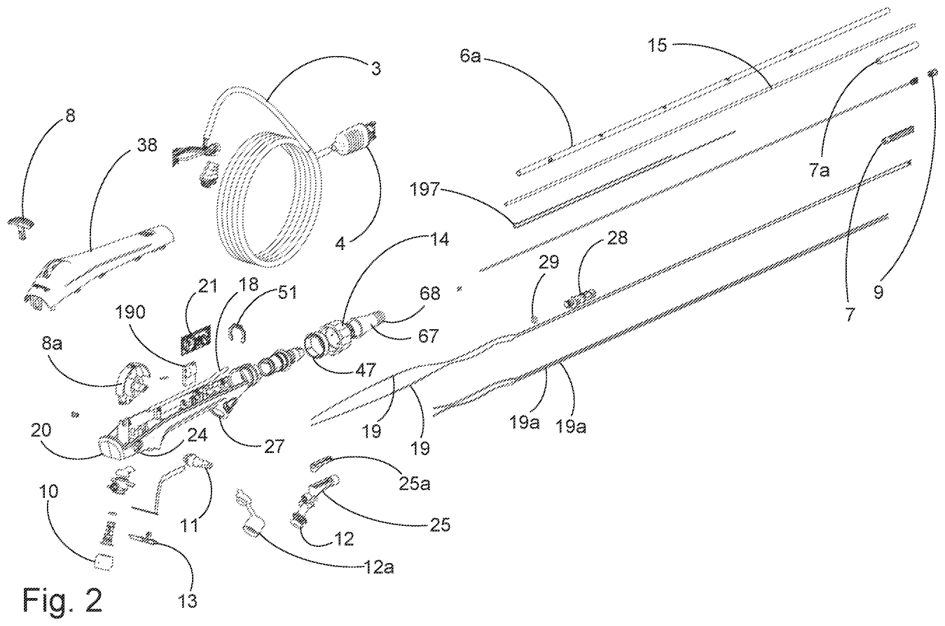

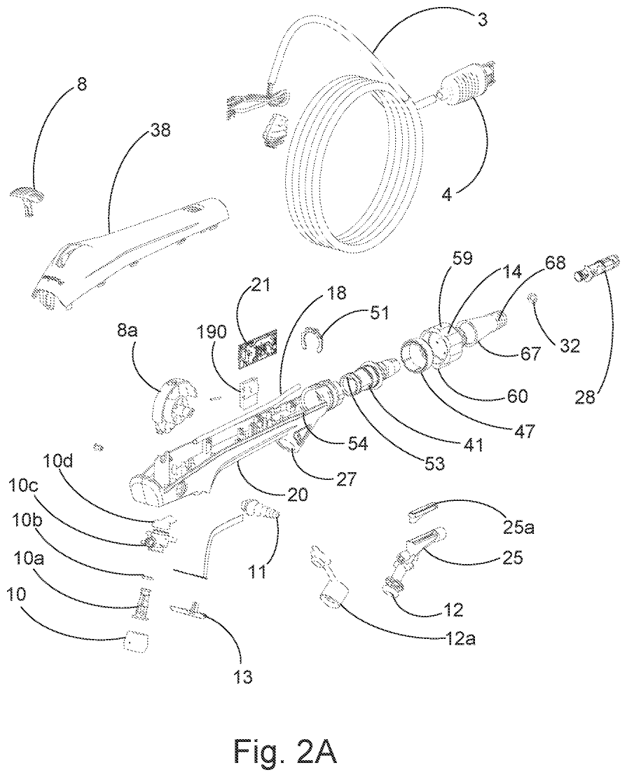

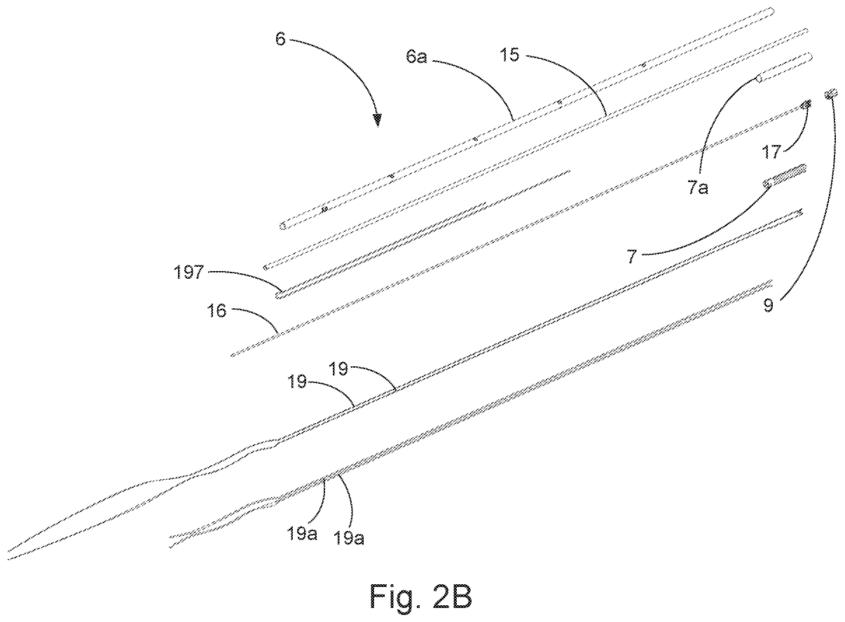

Turning first to FIG. 1, a system comprising an endoscope 1 and a display unit 2 is shown. The endoscope 1 may be connected to the monitor 2 by means of a cable 3 with a suitable connector 4. The endoscope 1 is preferably disposable i.e. intended to be thrown away after use on one single patient, whereas the display device 2 may be used multiple times with different reusable endoscopes. The endoscope is an insertion endoscope comprising a handle 5 at the proximal end, and an insertion cord 6 extending from the handle 5 towards the distal end of the endoscope 1. At the distal end of the insertion cord 6 a bending section, which may be controlled by an operating member 8 is provided. The insertion cord 6 also includes main tube 6a extending between the handle 5 and a bending section body 7. The bending section comprises the bending section body 7 (shown in FIGS. 2 and 2B) beneath a flexible cover 7a. In turn, at the distal end of the bending section body 7, a tip housing 9 with the image capture device, lenses, illumination, the distal port of a working and/or suction cannel etc. of the endoscope is provided. At the distal end, the working channel will normally serve dual functions as both working channels for tools and suction channel. The handle 5 normally also comprises a suction activation button 10 for activating the suction though the suction channel to an external vacuum source (not shown) via a suction connector 11. The handle may also have a tool insertion port 12 (cf. FIG. 2) with a cap 12a for the insertion of an external tool through the handle and the working channel so as to emerge from the distal working channel port at the tip housing 9. Also, the handle 5 may be provided with buttons for activation of electrical switches 13 controlling functions of the image capture device, such as taking of still images. In accordance with the present disclosure, the insertion cord 6 may be rotated with respect to the handle 5 by manually gripping and turning a gripping surface 14 associated with a coupling mechanism which will be describe below. The handle 5 comprises a handle housing part 20 and a lid 38.

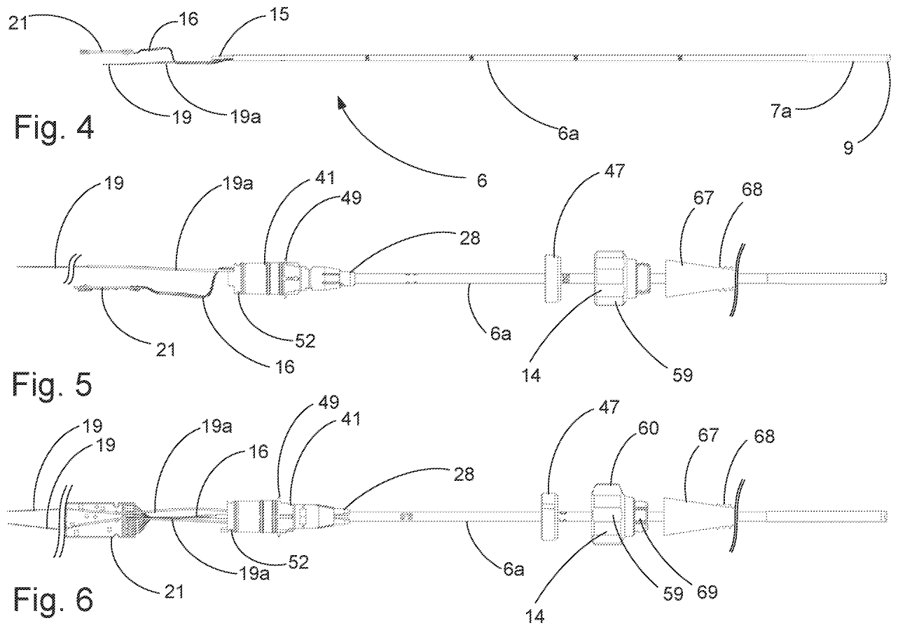

Turning now to FIGS. 2, 2A and 2B, the components of the exemplary embodiment of the endoscope 1 are shown. FIG. 2 is an exploded view including proximal components and distal components of the endoscope. In FIG. 2A, the components of the handle 5, the coupling mechanism and the connection cable 3 are shown. In FIG. 2B, the components of the insertion cord sub-assembly are shown. These include the tip housing 9 attached to the bending section body 7, which is in turn connected to the main tube 6a forming the majority of the body of the insertion cord 6. Inside the main tube 6a a working channel tube 15 is provided. The working channel tube 15 passes through the bending section body 7 and is attached to the tip housing 9, more specifically so attached that it is in communication with a passage through the tip housing 9 to a distal working channel port. Electrical wires 16, electrically connected to the image capture device 17, also run along the inside of the main tube 6a, through the bending section body 7 and into the tip housing 9 where the image capture device 17 is accommodated. Likewise, the supply cables for the illumination components, such as LEDs, run along the inside of the main tube 6a, through the bending section body 7 and into the tip housing 9 where also the illumination components are accommodated. Similarly, the pull wires 19 for bending the bending section body 7 run along the inside of the main tube 6a and through the bending section body 7. To avoid ingress of water or the like, the bending section body 7 is covered by a flexible cover 7a connected to the tip housing 9 and the main tube 6a in a sealed manner. A stabilizer 197 may also be accommodated in the main tube 6a. All these parts may be preassembled and the electrical wires 16 and supply wires preferably also connected to a circuit board 21, as shown in FIG. 4, to provide a pre-assembled sub-assembly to be joined with the handle 5, in a subsequent assembly step when assembling the endoscope 1. As can be seen, the electrical wires 16, supply cables and pull wires 19, as well as the printed circuit board 21, extend from the sub-assembly. Accordingly, the handle 5 and the coupling mechanism according to the disclosure have been adapted for easy assembly despite these extending parts.

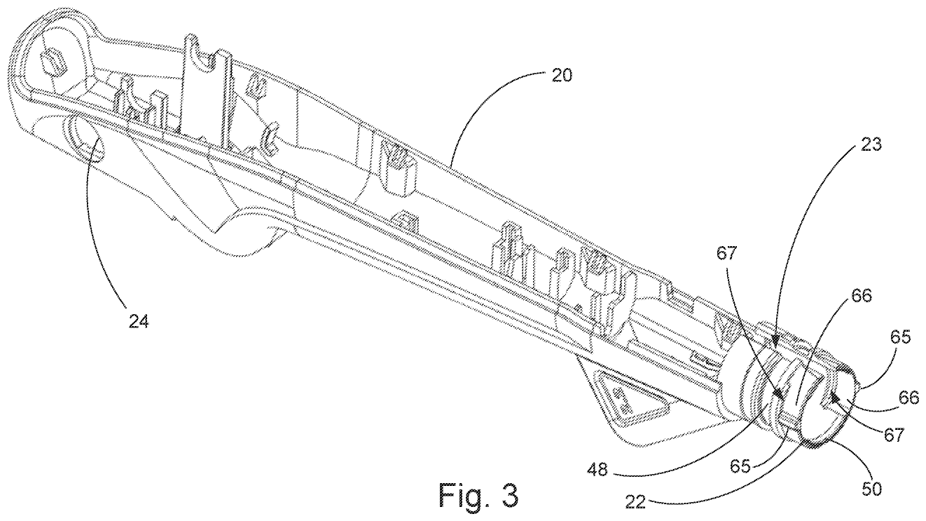

Turning now to FIG. 3, the handle housing part 20 is shown. The handle housing part 20 comprises a shell shape adapted for accommodating internal parts of the handle and is covered by the lid 38, cf. e.g. FIG. 2A, which is also somewhat shell-shaped. The handle housing part 20 serves as a base for supporting most of the internal parts accommodated in the handle 5, whereas the lid 38 essentially only serves as a closing cover. That is to say, the cover holds the operating member 8a, the anchoring block 190 for the guide tubes guide tubes 19a, the circuit board 21 and the C-shaped clip 51 in position, but does preferably not carry or support any internal parts apart from that. As can be seen, the distal handle housing end 22 is C-shaped in circumference, with a longitudinal slit 23. As will be elaborated below, the distal handle housing end 22 is adapted to form part of a rotary coupling mechanism allowing the insertion cord 6 of the insertion cord sub-assembly (shown in FIG. 4) to be turned with respect to the handle 5. The handle housing part 20 also comprises an opening 24 adapted to receive the suction connector 11 for connecting the partially combined suction and working channel to an external vacuum source. The partially combined suction and working channel comprises a Y-junction body 25 comprising a tool entry port 12 located in a further opening 27 in the handle housing part 20. A further opening (not visible) may be provided in the handle housing part 20 to accommodate a suction activation valve assembly 10a, 10b, 10c, 10d, associated with the suction activation button 10 and connected to the suction connector 11. The suction activation valve assembly 10a, 10b, 10c, 10d is furthermore connected to one end of an intermediate suction tube 18, the other end of the intermediate suction tube 18 being connected to the Y-junction body 25, e.g. in a receptacle 198 formed in one branch of the Y-junction body 25, as indicated in FIG. 9. The Y-junction body 25 may be integrally formed but for manufacturing reason is preferably provides in two pieces, i.e. including a Y-junction lid 25a, with a guiding surface 25b facilitating the insertion of a tool from the tool port.

The working channel tube 15, located in the centre of the main tube 6a, needs to be rotatable with respect to the distal end of the Y-junction body 25, which is fixed withing the handle 5. Pull wires 19, at least partially, need to be located off-centre to function. In any case, pull wires 19, electrical wires 16 and supply cables for the LEDs cannot coincide with the working channel tube 15, and therefore must also be accommodated off-centre. Electrical wires 16, supply cables and pull wires 19 may instead be made with overlength and slack so as to be able to twist and wrap. Accordingly, the pull wires 19 are preferably in guide tubes 19a to provide Bowden cables. To accommodate for all these requirements, the present disclosure utilises a connection member 28.

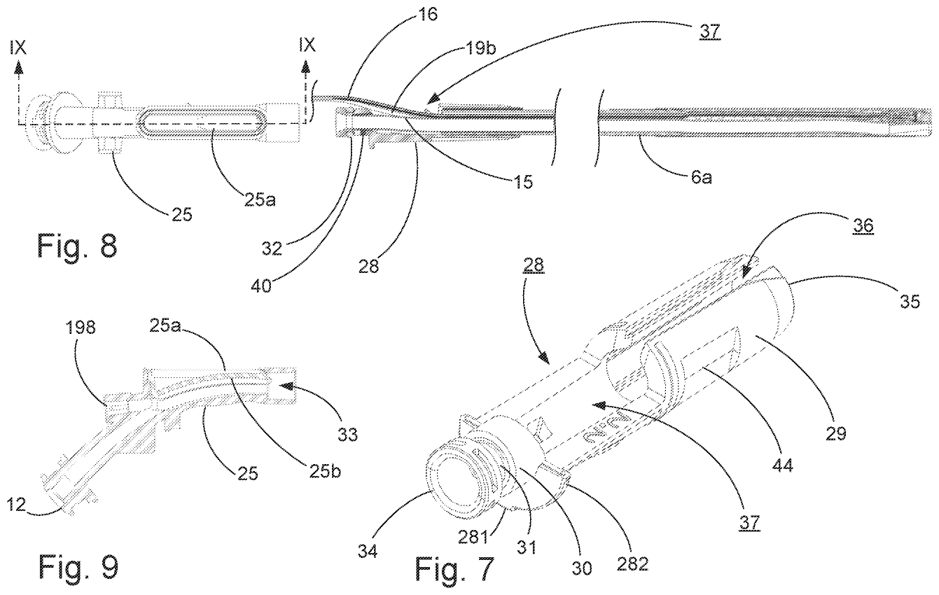

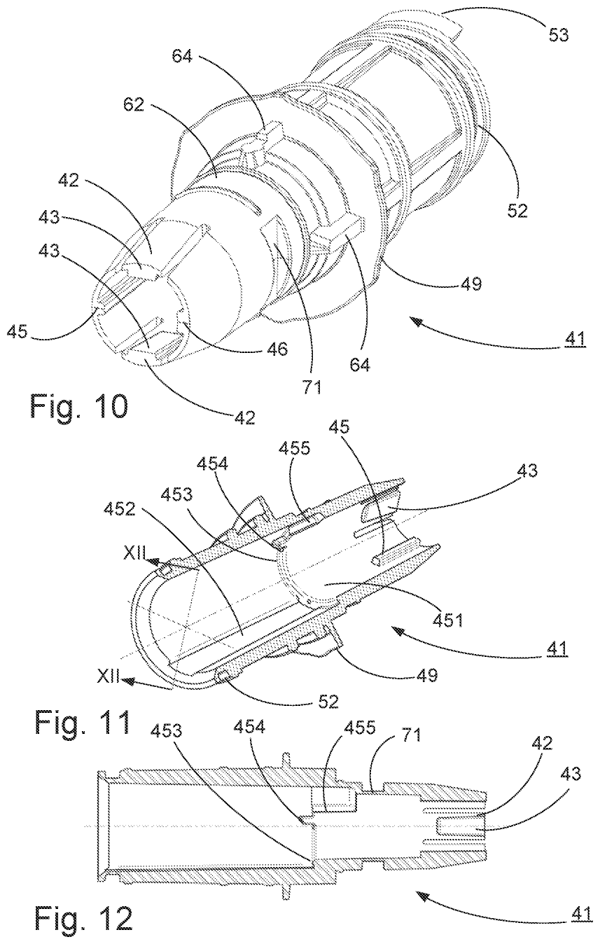

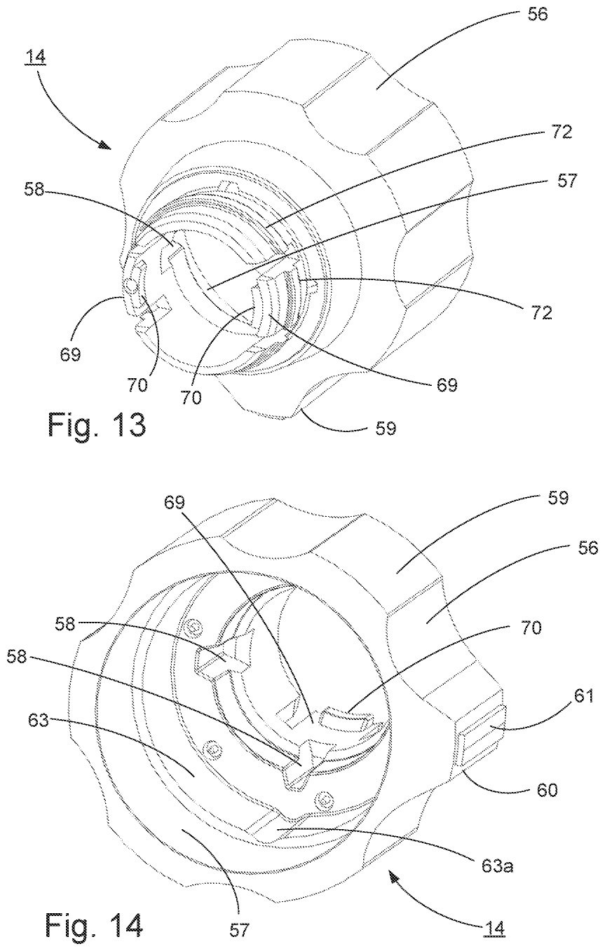

FIG. 7 shows the connection member 28 with a wall 29. At the proximal end, the connection member 28 comprises a section 30 where the wall is circumferentially closed. In this section 30, an outer circumferential recess 31 adapted to accommodate the sealing member 32, preferably in the form of an O-ring of an elastomer material, is provided. The outer diameter of the uncompressed sealing member 32 is slightly larger than the inner diameter of a cylindrical bore 33 provided in the distal end of the Y-junction body 25, so as to provide a seal allowing the connection member 28 to rotate with respect to the Y-junction body. The remainder of the wall 29 towards the distal end of the connection member 28 is not circumferentially closed. The wall 29 defining a through, but not circumferentially closed, passage between the proximal, first, end 34 and a distal, second, end 35. Towards the distal, second, end 35 the connection member 35 comprises a slit 36 in the wall 29. Between the slit 36 and the circumferentially closed section 30 of the wall 29 a larger opening 37 covering mostly half of the circumference is provided. As can be seen from the partial cross section of FIG. 8, this larger opening serves as an exit opening for the electrical wires 16, supply cables and pull wires 19 covered with the guide tubes 19a.

During assembly of the endoscope the main tube 6a is inserted into the passage of the connection member 28 to the position shown in FIG. 8 where it is secured, preferably my means of an adhesive. During this insertion, the electrical wires 16, supply cables and pull wires 19 extending from the proximal end of the main tube 6 of the insertion cord assembly may easily be passed through the slit 36 so as to emerge laterally from the exit opening 37, so that they will in the fully assembled endoscope pass around the outside of the Y-junction body inside the handle 5. This can be done even if the electrical wires 16 and supply cables have been joined to the printed circuit board 21 in the pre-fabrication of the insertion cord assembly, because the circuit board need not pass through the connection member 28, and the electrical wires 16 and supply cables need only pass the slit 36. The proximal end of the working channel tube 18 is adhered to the inside of the section 30 where the wall is circumferential, so as to provide a sealed connection. For this, a glue hole 40 for introducing the adhesive may preferably be provided in the otherwise circumferential wall section 30.

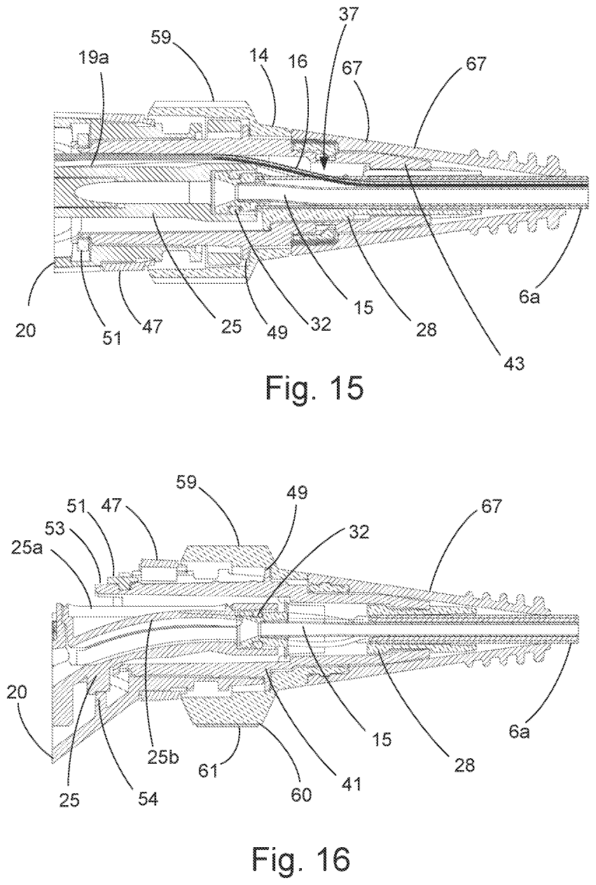

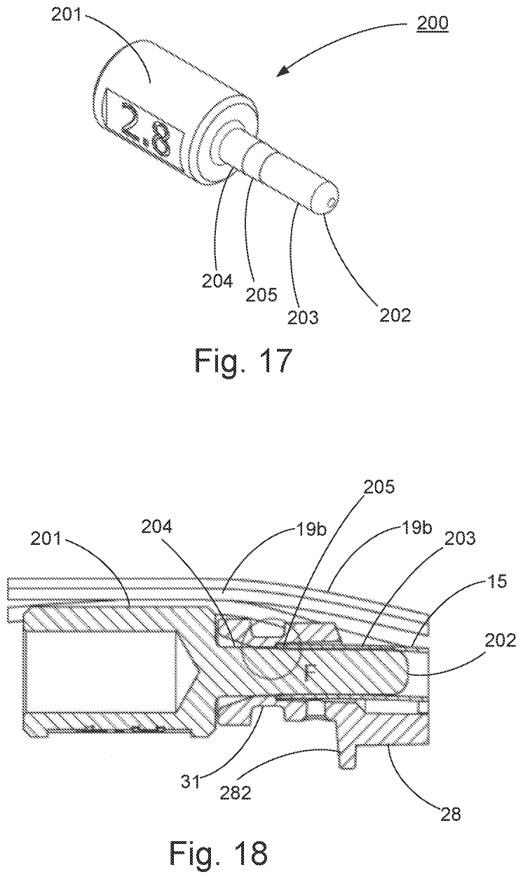

In order to ensure a smooth transition from the connection member 28 to the working cannel tube 15 the working channel tube 15 is preferably flared out at the proximal end. The smooth transition without edges is desirable to prevent procedure tools or optical fibres inserted through the Y-junction body 25 into the working channel 15 to be caught and disturbing the insertion process. For this a tool 200 is shown in FIGS. 15 and 16. The tool has a gripping part 201 for digitally gripping it with thumb, index finger and possibly middle finger when inserting it into the connection member 28 and the working cannel tube 15 as shown in FIG. 16. The gripping portion 201 is preferably knurled or in other way prepared for good grip. The tool furthermore comprises an insertion section with a rounded distal insertion end 202, a first cylindrical part 203 of a first diameter and a second cylindrical part 204 of second diameter. The first diameter is larger than the second diameter. A tapering section 205, e.g. frusto-conical is provided between the second cylindrical part 204 and the first cylindrical part 203.

As can be seen from FIG. 16, the frusto-conical section of the tool expands the working channel tube 15 and provides a flare in the transition area marked (with the circle F) which is maintained once the glue 40 has set. It also helps aligning the working channel tube with the bore of the connection member 28.

The connection member 28 may then be inserted into a bushing 41. The bushing is made of a low friction material such as POM. This is done by sliding the busing 41 over the insertion cord sub-assembly from the distal end and onto the connection member 28 where it clicks into position, as seen in FIGS. 4 and 5. POM is difficult to get adhesive to stick to and accordingly the connection member 28 and the bushing 41 comprise mutually interlocking features securing them with respect to each other in both the circumferential direction as well as in the longitudinal direction. Thus, as can be seen from FIG. 10 the distal end of the bushing 41 comprises two resilient arms 42 with inwardly extending protrusions 43 adapted to elastically snap into engagement with recesses 44 in the outer surface of the connection member 28, thus mutually securing the connection member 28 and the bushing 41 with respect to another. To ensure the correct orientation in the circumferential direction, i.e. ensuring that electrical wires 16, supply cables and pull wires 19 are later at the correct side within the handle ribs, the inner bushing wall surface 451 is provides with a first rib 45 and a second rib 46 of differing widths, where the first rib 45 is adapted to engage in the slit 36 in the connection member 28 and the second rib 46 is adapted to engage into a groove preferably arranged diametrically opposite side of slit in the outer surface of the connection member 28 (not visible in the figures). This also further secures the connection member 28 and the bushing with respect to another in the circumferential direction.

To end correctly up in this correct orientation in the circumferential direction, the proximal part of the inner bushing wall surface 451 comprises a longitudinal groove 452 adapted to guide a protrusion 281 on a flange 282 on the connection member 28, cf. FIG. 7. When sliding the bushing 41 over the connection member the flange 282 abuts a stop wall 453 formed by a step covering a majority of the circumference of the inner wall surface 451. The position of this stop wall 453 in the longitudinal direction is so selected that the inward extending protrusions 43 latch into the recesses 44 and secure the mutual position of the connection member 28 and the bushing 41 in the longitudinal direction. At the end of the stop wall 453 small protrusions 454 in the longitudinal direction are provided, so as to secure further the position of the connection member 28 with respect to the bushing 41 in the circumferential direction. For the remainder of the circumference, i.e. the minority part between the protrusions 454 the step is missing, so as to provide lateral space 455 for the wires protruding from the opening 37 in the connection member 28 in the assembled state.

The printed circuit board 21 with electrical wires 16, supply cables and pull wires 19 emerging from the opening 37 into the lateral space 455 and out of the proximal end of the bushing 41 may then be placed in housing part 20 using the slit 23 to pass the cables. A marker ring 47 may be slid over the end of the housing part 20, snap into a recess 48 and partially close the slit 23 at the distal end of the housing part. More specifically the marker ring 47 may be slid from the distal end of the insertion cord sub-assembly over the bushing 41 to surround the coupling mechanism and preventing expansion of the slit 23 by forces from the busing 41, thus later aiding in ensuring the desired amount of friction between the handle housing part 20 and the bushing 41.

The bushing 41, now connected to the insertion cord sub-assembly may then be inserted into the distal end of the handle housing now partially closed with the marker ring 47. In this position the bushing is secured against axial movement by a flange 49 provided on the outer bushing wall surface 62, and engaging the distal end surface 50 of the housing part 20, and a C-shaped clip 51 engaging in a circumferential recess 52 in the outer surface of the bushing 41. Protruding from the proximal end of the bushing 41 a stop member 53 is preferably provided. This stop member 53 is adapted to engage suitable uprights 54 on the interior surface of the housing part 20 to limit the rotation of the bushing 41 with respect to the handle 5. The uprights 54 may preferably also serve as anchoring points for the Y-junction body 25, i.e. points for adhering the Y-junction body 25 to the interior of the housing part 20.

As mentioned, the bushing 41 is preferably made from a low friction material. To provide for good grip for the operator needing to turn the bushing 41 and the insertion cord 6 with respect to the handle 5, an annular gripping member, or gripping ring, 14 is provided. The annular gripping member 14 has an external circumferential gripping surface 56 and an inner gripping member surface 57. Preferably, the annular gripping ring 14 comprises a number of teeth 59, 60, of which one tooth 60, preferably, extends radially outwardly more than the others and/or is provided with additional surface features 61 to provide the user with a sense of position. Like the other parts, the annular gripping member 14 is slid over the insertion cord sub-assembly from the distal end thereof.

The inner surface is preferably provided with longitudinal grooves 58 adapted to match external ribs 64 on the outer surface wall 62 of the bushing 41, so as to ensure their correct mutual orientation when assembling the endoscope 1, e.g. so as to have the tooth 60 extending correctly when in a neutral position with respect to the handle 5. Thus, at least one first inner gripping member surface part in the form of the longitudinal grooves 58 is providing engagement with said outer bushing wall 62.

Furthermore, a second inner gripping member surface wall part 63 is rotatably engaging the exterior of said housing part 20. The second inner gripping member surface part 63 comprises at least one, but preferably two, position reference members, e.g. in the form of a notch 63a adapted to mutually engage complementary position reference members 65 on the exterior of the housing part 20. Alternatively, the position reference member 65 on the exterior of the housing part 20 could be provided as a notch. The two position reference members are preferably located with a 180° spacing, in particular with 90° spacing from the tooth 60 on either side thereof. Similarly, the complementary position reference members 65 are located with a 180° spacing on either side of the housing part 20, in particular with 90° spacing from the slit 23 on either side thereof. The complementary position reference members 65 are preferably provided on leaf spring members 66 provided at the distal end of the housing part 20. The leaf spring members 66 may conveniently be formed integrally with the housing part 20 by provision of suitable circumferential slits 67 in the wall of the housing part 20. When aligned the reference members with their complementary parts, under the spring force from the leaf spring members, keeping insertion cord 6b in position with respect to the handle 5 until a user's attempt to turn the insertion cord 6b provides a force overcoming the forces of the leaf spring members 66.

To axially secure the annular gripping member 14 with respect to the bushing 41, the distal end of the annual gripping member 14 comprises a pair of elastic latching members 69 with inward protrusions 70 adapted to engage a corresponding pair of recesses 71 in the outer surface of the bushing 41.

Finally, to complete the assembly of the insertion cord sub-assembly to the handle 5 via the rotary coupling mechanism, a tension relief member 67 may be slid over the insertion cord 6 from the distal end thereof an onto the annular gripping member 14 to engage a generally circumferential ribs 72 at the elastic retaining members 69 on the distal end of the annular gripping member 14. The tension relief member 67 not only serves as tension relief but inter alia serves as sealing cover for the coupling mechanism and as a holding means for e.g. an endotracheal tube during intubation of a patient or for a protective tubular cover during storage of the endoscope 1, which may be held by a number of circumferential elastic ribs 68.

In this way a rotary coupling mechanism for an endoscope 1 has been provided in a manner easy to manufacture and easy to assemble.

We claim:

1-8. (canceled)

9. An endoscope comprising:
a handle comprising a housing part and an inner handle surface, the housing part comprising an exterior;
a working channel part within said handle;
a main tube;
a working channel tube;
electrical wires; and
a rotary coupling rotatably coupling the handle and the working channel tube about a rotation axis, the rotary coupling comprising:
a connection member comprising a wall, a proximal end, a distal end, a through-passage between the proximal end and the distal end, a lateral exit opening in the wall, and a slit in the wall extending distally from the lateral exit opening, the proximal end being rotatably coupled to the working channel part; and
a bushing having a bushing through-aperture, an inner bushing wall surface, and an outer bushing wall surface, the connection member received in the bushing through-aperture, and the outer bushing wall surface engaging the inner handle surface in said handle.

10. The endoscope of claim 9, wherein the main tube is affixed to the distal end of the connection member.

11. The endoscope of claim 9, further comprising an image sensor and a circuit board connected to the image sensor via the electrical wires, wherein the main tube, the working channel tube, the electrical wires, the image sensor and the circuit board comprise an insertion cord sub-assembly, wherein the electrical wires extend through the main tube and through the lateral exit opening of the wall of the connection member with the circuit board positioned proximally of the lateral exit opening after assembly of the endoscope.

12. The endoscope of claim 9, the endoscope further comprising a marker ring, an image sensor and a circuit board, wherein the housing part comprises a distal end comprising a longitudinal slit and a circumferential recess, wherein the electrical wires comprise a proximal end and a distal end, the proximal end connected to the circuit board and the distal end connected to the image sensor, wherein the main tube, the working channel tube, the electrical wires, the image sensor and the circuit board comprise an insertion cord sub-assembly, wherein after assembly of the insertion cord sub-assembly the electrical wires extend through the main tube and through the lateral exit opening of the wall of the connection member with the circuit board spositioned proximally of the lateral exit opening and the image sensor positioned distally of the main tube, and wherein the marker ring is positioned in the circumferential recess and over the longitudinal slit to prevent expansion of the longitudinal slit.

13. The endoscope of claim 9, the endoscope further comprising an image sensor, a circuit board, an annular gripping ring, and a marker ring, wherein the housing part comprises a distal end comprising a circumferential recess and a housing part position reference member distal of the circumferential recess, wherein the electrical wires comprise a proximal end and a distal end, the proximal end connected to the circuit board and the distal end connected to the image sensor, wherein the electrical wires extend through the main tube and through the lateral exit opening with the circuit board spositioned proximally of the lateral exit opening and the image sensor positioned distally of the main tube, wherein the bushing further comprises a flange extending radially outwardly from the outer bushing wall surface, wherein the annular gripping ring comprises a distal gripping ring inner surface, a proximal gripping ring inner surface, and a gripping ring position reference member, wherein the distal gripping ring inner surface is in engagement with said outer bushing wall surface distally of the flange, the proximal gripping ring inner surface rotatably overlaps an exterior of the housing part proximally of the flange, and the gripping ring position reference member engages the housing part position reference member, and wherein the marker ring is positioned in the circumferential recess of the housing part to prevent expansion of the longitudinal slit.

14. The endoscope of claim 13, wherein the distal end of the housing part further comprises a longitudinal slit, and wherein the inner bushing wall surface comprises a rib protruding into the longitudinal slit.

15. The endoscope of claim 9, wherein the rotary coupling further comprises an annular gripping ring having an external gripping ring surface, a distal gripping ring inner surface in engagement with said outer bushing wall surface, and a proximal gripping ring inner surface rotatably overlapping said exterior of the housing part.

16. The endoscope of claim 9, wherein the housing part comprises a distal end comprising a longitudinal slit and a circumferential recess, wherein the endoscope further comprises a marker ring positioned in the circumferential recess and over the longitudinal slit to prevent expansion of the longitudinal slit.

17. The endoscope of claim 16, wherein the inner bushing wall surface comprises a rib protruding into said longitudinal slit.

18. The endoscope of claim 9, wherein the bushing further comprises a flange extending radially outwardly from the outer bushing wall surface, wherein the rotary coupling further comprises an annular gripping ring having an external gripping ring surface, a distal gripping ring inner surface in engagement with said outer bushing wall surface distally of the flange, and a proximal gripping ring inner surface rotatably overlapping the exterior of the housing part proximally of the flange.

19. The endoscope of claim 18, wherein the distal gripping ring inner surface comprises a position reference member configured to mutually engage a complementary position reference member on the exterior of said housing part.

20. The endoscope of claim 19, wherein the housing part comprises, at a distal end thereof, a spring member extending in a circumferential direction, wherein the complementary position reference member is provided on the spring member.

21. The endoscope of claim 9, further comprising a locking clip securing said bushing against axial motion with respect to the housing part along said rotation axis.

22. The endoscope of claim 21, wherein the locking clip is made from the same material as the bushing.

23. A system comprising:
a display device; and
the endoscope of claim 9, the endoscope being connectable to said display device.

24. A method of making the endoscope of claim 9, the method comprising:
passing the electrical wires through the main tube;
connecting the electrical wires to an image sensor and to a circuit board, the circuit board extending proximally from the main tube and the image sensor extending distally from the main tube, after said connecting the electrical wires, the main tube, the image sensor and the circuit board forming an insertion cord sub-assembly;
after said connecting, passing the electrical wires throught the slit of the connection member; and
after passing the electrical wires throught the slit, affixing the main tube to the connection member.

25. The method of claim 24, the method further comprising sliding a bushing over the insertion cord sub-assembly from a distal end of the insertion cord sub-assembly and securing the bushing to the connection member via mutually interlocking features of the bushing and the connection member.

26. An endoscope comprising:
a handle comprising a housing part and an inner handle surface, the housing part comprising an exterior, and a distal end comprising a longitudinal slit and a circumferential recess;
a working channel part within said handle;
a main tube;
a working channel tube;
a marker ring;
an image sensor;
a circuit board;
electrical wires comprising a proximal end and a distal end, the proximal end connected to the circuit board and the distal end connected to the image sensor;
a rotary coupling rotatably coupling the handle and the working channel tube about a rotation axis, the rotary coupling comprising:
a connection member comprising a wall, a proximal end, a distal end, a through-passage between the proximal end and the distal end, a lateral exit opening in the wall, and a slit in the wall extending distally from the lateral exit opening, the proximal end being rotatably coupled to the working channel part; and
a bushing having a bushing through-aperture, an inner bushing wall surface, and an outer bushing wall surface, the connection member received in the bushing through-aperture, and the outer bushing wall surface engaging the inner handle surface in said handle,
wherein the electrical wires extend through the main tube and through the lateral exit opening of the wall of the connection member with the circuit board positioned proximally of the lateral exit opening and the image sensor positioned distally of the main tube, and
wherein the marker ring is positioned in the circumferential recess and over the longitudinal slit to prevent expansion of the longitudinal slit.

27. The endoscope of claim 23, wherein the inner bushing wall surface comprises a rib protruding into said longitudinal slit.

28. The endoscope of claim 24, the endoscope further comprising an annular gripping ring having an external gripping ring surface, a distal gripping ring inner surface in engagement with said outer bushing wall surface distally of the flange, and a proximal gripping ring inner surface rotatably overlapping the exterior of the housing part proximally of the flange, and
wherein the distal gripping ring inner surface comprises a position reference member configured to mutually engage a complementary position reference member on the exterior of said housing part.
